(19) United States
(12) Patent Application Publication
(10) Pub. No.: US 2004/0139489 A1
(43) Pub. Date: Jul. 15, 2004

(54) Method for generating cloned animals using chromosome shuffling

(76) Inventors: Robert Lanza, Clinton, MA (US); Jose Cibelli, Holden, MA (US); Philip Damiani, Spencer, MA (US); Michael D West, Boston, MA (US)

Correspondence Address: CROWELL & MORING LLP, INTELLECTUAL PROPERTY GROUP, P.O. BOX 14300, WASHINGTON, DC, 20044-4300, US

(21) Appl. No.: 10/398,614

(22) PCT Filed: Oct. 5, 2001
(86) PCT No.: PCT/US01/31216

(51) Int. Cl. C12N 15/00; A01K 67/027

(52) U.S. Cl. 800/21

(57) ABSTRACT

The present invention concerns the use of chromosomal replacement techniques in the context of producing cloned and transgenic animals, in order to correct chromosome abnormalities or alter autosomal genotypes, and provide for novel breeding pairs by replacing the sex chromosome in animals to be cloned. Replacement of a sex chromosome, or an X or Y chromosome, will result in animals that are autosomally isogenic and sexually non-isogenic (AISN), with “autosomally isogenic” meaning that the paired sets of autosomes (non-sex chromosomes) in each animal are isogenic or identical. Also included in the invention are animals that are both “autosomally” and “allelically” isogenic whereby each particular pair of chromosomes is internally isogenic or identical within a single animal as well as between animals. Such animals are particularly useful in generating a line of cloned mammals using sexual reproduction, without having to undergo nuclear transfer in order to propagate cloned animals.

### CROSS REFERENCE TO RELATED APPLICATIONS

This application claims priority from U.S. Provisional Application Serial No. 60/238,014, filed Oct. 6, 2000, which is incorporated herein in its entirety.

### FIELD OF INVENTION

This invention concerns methods of cloning animals that incorporate methods for manipulating or shuffling chromosomes. The methods find important use in the fields of agriculture, xenotransplantation, laboratory science and species conservation, where shuffling of chromosomes can be used to correct chromosomal abnormalities, and to create autosomally isogenic, sexually non-isogenic cloned animals.

### BACKGROUND OF THE INVENTION

Microcell-mediated chromosome transfer has been used for many years in order to introduce a single chromosome into a target cell. For instance, in 1986, Saxon et al demonstrated the complete suppression of tumorigenicity in HeLa cells by introducing a single human chromosome via microcell fusion. See Saxon et al., 1986, “Introduction of human chromosome 11 via microcell transfer controls tumorigenic expression of HeLa cells,” EMBO J. 5: 3461-66. This technology was used by others to create libraries of human chromosomes, by fusing single human chromosomes carrying the HGPRT gene with HGPRT-deficient mouse cells. See Koi et al., 1989, “Construction of mouse A9 clones containing a single human chromosome (X/autosome translocation) via Micro-cell fusion,” Jpn. J. Cancer Res. 80: 122-25. Other investigators have also used microcell fusion to demonstrate that tumorigenicity of various tumor cell lines is suppressed by introducing a single human chromosome. See Yoshida et al., 1994, “Alteration of tumorigenicity in undifferentiated thyroid carcinoma cells by introduction of normal chromosome 11,” J. Surg. Oncol. 55:170-74; see also Dong et al., 1996, “Prostate cancer—biology of metastasis and its clinical implications,” World J. Urol. 14: 182-89. However, there have been no reports of the use of microcell mediated fusion for the replacement of chromosomes in cells and animals, or the simultaneous removal of chromosomes in addition to microcell-mediated chromosome transfer.

Other techniques have been used to replace entire chromosomes or very large fragments of chromosomes. For instance, U.S. Pat. No. 5,721,367 by Kay et al describes methods for replacing greater than 50 kb of a mammalian genome, and transgenic mammals comprising >50 kb transgenes integrated into their genome. However, 50 kb is only a small fraction of the length of a typical mammalian chromosome, and such replacements will not result in substitutions of entire chromosomes, or correction of chromosomal defects that are at opposite ends of a chromosome. U.S. Pat. No. 6,077,697 by Hadlaczky and Szalay describes mammalian artificial chromosomes (MACs) which are stable and self-replicating, and may be used to permit targeted integration of megabase pair size DNA fragments. However, MACs do not themselves replace native chromosomes or correct existing chromosomal defects. Thus, it would be advantageous to have a system for replacing entire chromosomes in mammalian cells, particularly in the context of cloned and transgenic mammals.

The recent showing that somatic cells may be used as donors for nuclear transfer enables the development of complex genetic manipulations in the context of cloning that were not considered possible before. For instance, experiments performed in the early 1990's suggested that when an embryo progresses to the blastocyst stage (the embryonic stage where the first two cell lineages separate) the efficiency of nuclear transfer decreases dramatically. See Collas and Robl, 1991, “Relationship between nuclear remodeling and development in nuclear transplant rabbit embryos,” Biol. Reprod. 45: 455-465. For example, inner cell mass cells (cells from the blastocyst which form both somatic and germ cells) were found to support a low rate of development to the blastocyst stage with some offspring obtained. See Collas and Barnes, 1994, “Nuclear transplantation by microinjection of inner cell mass and granulosa cell nuclei,” Mol. Reprod. Devel. 38: 264-67; see also Sims et al., 1994, “Production of calves by transfer of nuclei from cultured inner cell mass cells,” Proc. Natl. Acad. Sci. USA 91: 6143-47. However, it was found that trophectodermal cells (the cells from the blastocyst that form the placenta) did not support the development of the nuclear fusion to the blastocyst stage. Collas and Robl, 1991. Based on these observations, as well as early experiments with amphibian nuclear transplantation, it was the overwhelming opinion of those skilled in the art at the time that once a cell becomes committed to a particular somatic cell lineage, its nucleus irreversibly loses its ability to be “reprogrammed,” i.e. to support full term development when used as a nuclear donor for nuclear transfer.

Therefore, it was quite astounding in 1998 when researchers at the Roslin Institute reported that cells committed to somatic cell lineage could support embryo development when used as nuclear transfer donors. Equally astounding, and more commercially significant, scientists at the University of Massachusetts and Advanced Cell Technology then showed the production of transgenic cattle by nuclear transfer using transgenic fibroblast donor cells. See also, Wells, 1998, “Cloning symposium: Reprogramming cell fate—transgenesis and cloning,” Monash Medical Center, Melbourne, Australia, April 15-16 (reporting the production of a calf using fibroblast cells). Differentiated cells have also been successfully used as nuclear transfer donors to produce cloned mice. See Wakayama et al., 1998, “Full-term development of mice from enucleated oocytes injected with cumulus cell nuclei,” Nature, 394: 369-74.

Still further, an experiment by researchers at the University of Massachusetts and Advanced Cell Technology was recently reported in a lead story in the New York Times, January 1999, wherein a nuclear transfer fusion embryo was produced by the insertion of an adult human differentiated cell (obtained from the cheek of an adult human donor) into an enucleated bovine oocyte. Thus, it would appear, based on these results, that at least under some conditions differentiated somatic cells can be reprogrammed or de-differentiated through the process of nuclear transfer.

It would be advantageous, therefore, if somatic cells to be used for nuclear transfer could be used to facilitate complex genetic manipulations of donor cells, and particularly the replacement of chromosomes in cloned animals.

### SUMMARY OF INVENTION

The present invention makes use of somatic cell donor cells for nuclear transfer to create complex chromosomal arrangements, and particularly chromosomal replacements, in cloned and transgenic animals. This technology may be used to produce cloned cells, embryos, blastocysts, fetuses and animals that are autosomally isogenic and sexually non-isogenic, in order to make a population more uniform or improve quality control in xenotransplantation. Such chromosome shuffling techniques can also be used to eliminate chromosomal abnormalities, such as inversions or translocations from the clone of an animal, produce a sexual mate for an extinct animal where the genome of only one animal is extant, or to produce the opposite sex of an existing animal or embryo where the genome of only one sex is available or desired.

For instance, when the object is to produce a female animal having desired traits, somatic cells from the desired animal are isolated, and one X chromosome is removed and replaced with a Y chromosome from another animal. Alternatively, both X chromosomes may be replaced with the sex chromosomes—one X and one Y—from another mammal. Multiple females may be cloned from the original somatic cells, and males would be produced from the autosomally isogenic sexually non-isogenic (AISN) cells. The cloned bulls could then be used to breed females by sexual reproduction rather than by cloning. Furthermore, semen from these males can be frozen for use in artificial insemination in order to produce more females having the desired trait.

To improve the effectiveness of this business model, the male AISN animals may be genetically modified to produce only female animals using the technology described in Application Serial No. 60/184,830, now PCT/US01/05932, herein incorporated by reference in its entirety. This would allow the marketing of semen and the ready propagation of female animals having a desirable genetic make-up while simultaneously preventing customers from breeding their the AISN animals on their own without exchange of compensation for the technology. In the case of the beef and pig industries, single sex technology can also be used to produce all male offspring, and female AISN animals could be marketed as a business strategy.

Normal sexual reproduction, however, results in crossing over of chromosomes and random segregation of alleles in the haploid gametes, and can lead to genetic diversity even in the offspring of autosomally isogenic cloned animals because these cloned animals still have two different chromosomal alleles in each pair of cloned chromosomes. Therefore, the chromosome shuffling techniques of the present invention may also be combined with nuclear transfer techniques designed to create homozygous diploids of desirable haploid genomes, in order to achieve allelically isogenic breeding pairs of animals that differ only as to their sex chromosomes, i.e., each is a complete autosomal homozygous diploid. Breeding autosomally and allelically isogenic animals results in isogenic male and female offspring without the need for years of inbreeding or successive cloning in order to generate animals. Further, such breeding avoids the potential genetic diversity associated with sexual reproduction between cloned breeding pairs where crossing over and chromosomal segregation can result in the appearance of undesirable recessive traits in the progeny.

Such allelically isogenic breeding pairs will have significant utility in the agricultural field where it is often desirable to propagate animals with specific traits such as high milk output, milk with specific lipid or protein profiles, or animals which produce meat, leather, wool or fiber having a desired characteristic. Such breeding pairs would also find utility in laboratory settings as well as xenotransplantation studies, where lowering the statistical “noise” from genetic diversity, or eliminating the risk of introducing viral contaminants is desirable. Autosomally and allelically isogenic breeding pairs provide the ultimate business model, whereby purchasers and handlers can be assured that desirable animals may be easily maintained via sexual reproduction or artificial insemination without the need for nuclear transfer techniques.

Thus, it is an object of the present invention to provide methods of altering the sex of a cloned animal, embryo, fetus or cell by removing or replacing one sex chromosome with the alternative sex chromosome from another animal.

It is also an object of the present invention to provide methods for producing a sexual mate for an extinct or endangered animal, where the alternative chromosome that is inserted may be from either a non-isogenic allogeneic animal or cell, or a xenogeneic animal cell, i.e., from a species closely related to the extinct animal, if there are no existing allogeneic mates.

Also provided are methods for eliminating chromosomal abnormalities from the clone of an animal, whereby damaged autosomes are removed and replaced with non-damaged autosomes from a non-isogenic animal.

Also provided are methods of making autosomally and allelically isogenic breeding pairs, whereby chromosomal shuffling and nuclear transfer are used to make haploid cells that can be combined or used in the G2 stage of the cell cycle to produce completely homozygous diploids that are sexually non-isogenic. Methods of making autosomally isogenic, allelically isogenic diploid nuclear transfer units are also encompassed, as are methods of making cells, oocytes, balstocysts, inner cell masses, ES cells, embryos, and individual animals having the same characteristics.

An object of the invention is also to provide business methods for using the breeding pairs to mass produce animals that have been genetically modified, bred or selected to provide an advantage in a desired market, as well as business methods to maintain control over the breeding of such animals by marketing animals and/or semen that can only be used by purchasers and handlers to produce animals of a single sex.

### BRIEF DESCRIPTION OF THE DRAWINGS

FIG. 1. In protocol for Percoll separation of somatic cells from semen, diagram depicting Percoll layers prior to (A) and following (B) centrifugation.

### DETAILED DESCRIPTION OF THE INVENTION

The present invention concerns the use of chromosomal replacement techniques in the context of producing cloned and transgenic animals, in order to correct chromosome abnormalities or alter autosomal genotypes, and provide for novel breeding pairs by replacing the sex chromosome in animals to be cloned. Replacement of a sex chromosome, or an X or Y chromosome, will result in animals that are autosomally isogenic and sexually non-isogenic (AISN), with “autosomally isogenic” meaning that the paired sets of autosomes (non-sex chromosomes) in each animal are isogenic or identical. Also included in the invention are animals that are both “autosomally” and “allelically” isogenic whereby each particular pair of chromosomes is internally isogenic or identical within a single animal as well as between animals.

The invention therefore encompasses methods of altering the sex of a cloned animal, or an animal to be cloned, or an embryo, blastocyst, fetus or cell comprising:

(1) isolating a somatic cell from an animal to be cloned;

(2) removing or programming for removal one sex chromosome from said somatic cell;

(3) inserting the alternative sex chromosome from a non-isogenic animal; and

(4) using nuclear transfer to create an autosomally isogenic, sexually non-isogenic animal, embryo, blastocyst, fetus or cell.

Such methods may also be used in instances whereby an offspring of a particular sex is desired as a result of sexual reproduction, where the method includes:

(1) isolating a fertilized ovum, embryo or blastocyst;

(2) testing the sex of said ovum, embryo or blastocyst;

(3) removing or programming for removal the sex chromosome from one cell of said ovum, embryo or blastocyst if it is not of the desired sex;

(4) inserting the alternative sex chromosome isolated from an allogeneic animal;

(5) using nuclear transfer to create an autosomally isogenic, sexually non-isogenic embryo or blastocyst; and

(6) implanting said embryo or blastocyst into a surrogate female to isolate an animal having a desired sex.

When a sex chromosome is removed according to the present invention, it may be either an X or a Y chromosome, and it may be replaced by the alternative sex chromosome from a non-isogenic allogeneic animal, or even a non-isogenic, xenogeneic animal. In the case where the somatic cell of interest is from a male animal, the Y chromosome may be replaced by the X chromosome from another copy of the somatic cell to yield a cell with two X chromosomes.

Also encompassed are methods of producing a sexual mate for an extinct or endangered animal by removing or programming for removal one sex chromosome from said somatic cell and inserting the alternative sex chromosome from a non-isogenic animal, and using nuclear transfer to create an autosomally isogenic, sexually non-isogenic animal mate for an extinct or endangered animal. In this embodiment, particularly for extinct animals, the somatic cell may need to be isolated from a sample of frozen cells. In cases where an animal is endangered or nearing endangered levels, somatic cells, preferably semen cells, may be frozen in preparation for the methodology of the invention. Where the animal is extinct and frozen cells for replacement chromosomes do not exist, the alternative chromosome may be taken from a xenogeneic animal, preferably one that is closely related to the extinct animal. In this regard, application Ser. No. ______ pertains specifically to the cloning of endangered species, which material is hereby incorporated in its entirety.

Also encompassed are methods of eliminating chromosomal abnormalities from the clone of an animal a damaged chromosome from a somatic cell is removed or programmed for removal, and a non-damaged chromosome from a non-isogenic animal is inserted. Nuclear transfer is then used to create an animal, embryo, blastocyst, fetus or cell from said chromosomally corrected somatic cell.

The chromosome to be replaced may be removed by any feasible technique. For instance, the unwanted chromosome may be removed by targeting by homologous recombination a gene or DNA sequence that results in loss of the chromosome upon mitosis or meiosis. As discussed in U.S. Pat. Nos. 5,270,201 and 6,077,697, chromosomal instability results when sequences are introduced which function as a centromere. Such sequences cause a dicentric chromosome to be created, which undergoes breakage potentially leading to loss of the chromosome during cell division. Loss of chromosomes that have been genetically modified with additional centromeric sequences can be detected by karyotype analysis. Cells which lose the targeted chromosome may be also be selected by including a negative selectable marker such as thymidine kinase whereby cells retaining the chromosome or pieces of the chromosome will not survive under selective conditions (i.e., gancyclovir in the case of thymidine kinase).

As noted above, an advantage of using somatic cells as nuclear donors is that they may be expanded readily in culture prior to chromosome shuffling techniques. However, embryonic cells may also be used, as may the nuclei of somatic cells, which are advantageous in that they may be preserved in a preservative (such as alcohol) prior to nuclear transfer, i.e., stored for future use. Preferred somatic cells will be proliferating, i.e., in a proliferative state, but need not necessarily be expanded in culture. The somatic cells may be genetically altered in other ways prior to or subsequent to chromosome exchange. For instance, said cells may be modified with a chromosomal insertion or deletion, where a transgenic animal is desired that produces specific proteins in its bodily fluids or mammary glands, or where it is desirable to remove or mutate genes involved in xenotransplantation rejection. The alternative sex chromosome to be introduced may also be genetically altered from its native state.

The methods of the present invention may be performed with a wide variety of animals, including mammals, fish, reptiles or birds. Preferred animals for agricultural and xenotransplantation uses to be made by the present invention are ungulates selected from the group consisting of bovine, porcine, sheep and goat. Preferred extinct or endangered animals to be reconstituted by the methods of the present invention include the gaur, bucardo, giant panda, cheetah, African bongo antelope, Sumarran tiger, Giant panda, Indian desert cat, mouflon sheep and rare red deer. Preferred animals to be generated for laboratory use include mouse, hamster, guinea pig and primates. The methods may also be used to clone cats, dogs, horses or other companion animal, or breed champion lines of such mammals.

The chromosomes to be inserted according to the claimed methods may be inserted via microcell-mediated chromsome transfer, or any other suitable technique known in the art, e.g., via injection. Methods for the preparation and fusion of microcells containing single chromosomes are well known. See, e.g., U.S. Pat. Nos 5,240,840; 4,806,476; 5,298,429 (herein incorporated by reference in their entirety; see also Fournier, 1981, Proc. Natl. Acad. Sci. USA 78: 6349-53; Lambert et al., 1991, Proc. Natl. Acad. Sci. USA 88: 5907-59; Yoshida et al., 1994, J. Surg. Oncol. 55:170-74; Dong et al., 1996, World J. Urol. 14: 182-89. Chromosomes to be introduced into cloned cells or cells to be cloned will preferably include a selectable marker, such as aminoglycoside phosphotransferase, for example, so that cells receiving the chromosome via microcell fusion may be readily selected from those that do not. In this regard, Siden and colleagues describe the construction of a panel of four microcell hybrids containing four separate insertions of the exogenous neomycin resistance gene into mouse chromosome 17. See Siden et al., 1989, Somat. Cell Mol. Genet. 15(3): 245-53.

U.S. Pat. No. 6,133,503 also describes methodology for producing microcells by treating a host donor cell with a mitotic spindle inhibitor such as colchicine, which results in the formation of micronuclei, then with cytochalasin B, which results in the extrusion of microcells which contain one or a few chromosomes. The methods of U.S. Pat. No. 5,635,376 are also helpful in the context of the present invention, in that this patent provides for female muntjac cell lines in which there is, for example, a ten-fold difference in chromosomal size between the diploid muntjac chromosomes and human chromosome 11. Thus, these female muntjac cell lines are useful for the amplification of desired chromosomes prior to use in cells to be cloned because desired chromosomes may be purified to apparent homogeneity from the resulting hybrids using conventional equipment given the large size difference between the chromosome of interest and the muntjac chromosomes. These patents are herein incorporated by reference in their entirety.

The cloned animals, embryos, blastocysts, fetuses and cells produced by the methods described herein are also part of the invention, as are the sexual mates and breeding pairs produced and their offspring. Also included are the individual replacement chromosomes used for the present invention and any DNAs used to make genetic modifications, as well as any intermediary cell lines such as muntjac cell lines used to amplify the desired replacement chromosomes.

As described briefly above, in certain embodiments, particularly business models where isogenic animals are to be produced via sexual reproduction or artificial insemination, it is desirable that the animals be allelically isogenic as well as autosomally isogenic. Accordingly, the present invention includes methods of making an autosomally isogenic, allelically isogenic breeding pair of animals comprising:

(1) isolating a somatic cell from a preferred animal;

(2) inducing meiosis to produce a haploid cell from said somatic cell;

(3) making a diploid cell from said haploid cell which contains isogenic alleles;

(4) expanding said diploid cell;

(5) isolating a copy of said diploid cell or the nucleus therefrom;

(6) removing one sex chromosome from said copy of said isolated diploid cell;

(7) inserting the alternative sex chromosome from a non-isogenic animal;

(8) using nuclear transfer to create a first animal that is autosomally isogenic, allelically isogenic and sexually non-isogenic to said allelically isogenic diploid cell; and

(9) using nuclear transfer to create a second animal that is autosomally isogenic, allelically isogenic and sexually isogenic to said allelically isogenic diploid cell, wherein sexual reproduction between said first animal and said second animal produces offspring that are autosomally isogenic and allelically isogenic to said first and second animal.

Such methods may be further supplemented by ensuring that the breeding pair of animals only produces animals of a single sex, by also including a step or steps whereby a nucleic acid construct is introduced into at least one sex chromosome of the germ line of said male animal, wherein said nucleic acid construct encodes a transgene which is expressed post-meiotically in developing spermatids, and wherein expression of said transgene alters the fertility of sperm resulting from said developing spermatids, such that said male produces progeny of a single sex. Such methods are described in copending Application Serial No. 60/184,830, which is herein incorporated by reference in its entirety.

Inducing meiosis to produce a haploid cell from a somatic cell may be accomplished by any successful method. Preferably, meiosis is accomplished by nuclear transfer of said somatic cell or the nucleus from said somatic cell (2n) into a metaphase II enucleated oocyte, and activating said nuclear transfer unit to extrude a polar body (n), thereby resulting in a haploid activated nuclear transfer unit. Activation may be accomplished by exposing said nuclear transfer unit to one or more treatments selected from the group consisting of hyaluronidase, ethanol, cytochalasin B, Ca²⁺ ions, change in osmolarity, electrical pulse, bohemine, ionomycin and sperm factor. The fact that haploid oocytes, when activated, form morphologically normal blastocysts has been documented by several researchers. See Kaufman, 1982, J. Embryol. Exp. Morphol. 71: 139-54 (reporting activation with 7% ethanol); Mann and Lovell-Badge, 1984, Nature 310(5972): 66-7; O'Neill and Kaufmnan, 1988, 248(1): 125-31 (reporting activation with hyaluronidase); De Sutter et al., 1992, J. Assist. Reprod. Genet. 9(4): 328-37 (activation using puromycin); Henery and Kaufmnan, 1992, Mol. Reprod. Dev. 31(4): 258-63 (activation in 7% ethanol); Kim et al., 1997, Zygote 5(4): 365-70 (activation by ethanol plus cytochalasin B); Escriba and Garcia-Ximenez, 1999, Theriogenology 51(5): 963-73, and 2000, Anim. Reprod. Sci. 28: 59(1-2): 99-107 (activation by altering the osmolarity and Ca²⁺ concentration with electrical pulses in mannitol medium); and Alberio et al., 2000, Mol. Reprod. Dev. 55(4): 422-32 (reporting that bohemine with or without ionomycin produces activated haploid oocytes).

Diploid cells containing isogenic alleles may be made by allowing the activated haploid oocyte to develop to at least the two cell stage, isolating and/or separating the cells, and fusing two allelically isogenic haploid cells from said developing activated oocyte into an enucleated metaphase II oocyte. Alternatively, the homozygous diploid may be made by isolating one haploid cell and allowing it to advance to the G2 phase of the cell cycle, at which point it is 2n or transiently diploid, and may be used as the donor nucleus for nuclear transfer. Some researchers have used cytochalasin B to induce diploidization of a female pronucleus following removal of the male pronucleus from a fertilized egg. See Markert and Petters, 1977, “Homozygous mouse embryos produced by microsurgery,” J. Exp. Zool. 201(2): 295-302; see also Anderegg and Markert, 1986, “Successful rescue of microsurgically produced humozygous uniparental mouse embryos via production of aggregation chimeras,” Proc. Natl. Acad. Sci. USA 83(17): 6509-13. Kono and colleagues have also shown that heterozygous bispermic androgenones (eggs with two Y chromosomes made by fertilizing enucleated oocytes in vitro), also develop to the blastoyst stage. See Kono et al., 1993, Mol. Reprod. Dev. 34(1): 43-6.

Thus, specific methods of making allelically isogenic AISN breeding pairs according to the present invention include several embodiments. For instance, included are methods of making an autosomally isogenic, allelically isogenic breeding pair of animals comprising:

(1) isolating a somatic cell from a preferred female animal;

(2) inducing meiosis to produce a haploid cell from said somatic cell;

(3) expanding said haploid cell;

(4) isolating a copy of said haploid cell or the nucleus therefrom;

(5) removing the X chromosome from said copy of said isolated haploid cell;

(6) inserting a Y chromosome isolated from a male animal;

(7) using nuclear transfer to create a first male animal that is autosomally isogenic, allelically isogenic and sexually non-isogenic to said haploid cell by fusing an isolated haploid cell or the nucleus therefrom selected from the expanded haploid cells of step (3) and the haploid cell or the nucleus therefrom from the haploid cell of step (7) with an enucleated metaphase II oocyte;

(8) using nuclear transfer to create a second animal that is autosomally isogenic, allelically isogenic and sexually isogenic to said haploid cell, by fusing two isolated haploid cells or the nuclei therefrom selected from the expanded haploid cells of step (3) with an enucleated metaphase II oocyte;

wherein sexual reproduction between said first animal and said second animal produces offspring that are autosomally isogenic and allelically isogenic to said first and second animal.

Also included are methods of making an autosomally isogenic, allelically isogenic breeding pair of animals comprising:

(1) isolating a somatic cell from a preferred male animal;

(2) inducing meiosis to produce a haploid cell from said somatic cell;

(3) selecting a single haploid cell and determining whether it contains an X or Y chromosome;

(4) expanding said haploid cell;

(5) isolating a copy of said haploid cell or the nucleus therefrom;

(6) removing the sex chromosome from said copy of said isolated haploid cell;

(7) inserting the alternative sex chromosome into said copy of said haploid cell wherein the alternative sex chromosome is isolated from either a non-isogenic animal or the original preferred animal or another haploid cell produced from said somatic cell and optionally expanding said haploid copy if an X chromosome is inserted;

(8) using nuclear transfer to create two animals that are autosomally isogenic, allelically isogenic and sexually non-isogenic by fusing isolated haploid cells or the nuclei therefrom from the expanded haploid cells of step (4) and/or the haploid cell or cells or the nuclei therefrom from the haploid cell or cells of step (7) with an enucleated metaphase II oocyte in order to create one animal that has two X chromosomes and one animal that has an X and a Y chromosome;

wherein sexual reproduction between said first animal and said second animal produces offspring that are autosomally isogenic and allelically isogenic to said first and second animal.

Also included are methods of making an autosomally isogenic, allelically isogenic breeding pair of animals comprising:

(1) isolating a somatic cell from a preferred female animal;

(2) inducing meiosis to produce a haploid cell from said somatic cell;

(3) expanding said haploid cell;

(4) isolating a copy of said haploid cell or the nucleus therefrom;

(5) removing the X chromosome from said copy of said isolated haploid cell;

(6) inserting a Y chromosome isolated from a male animal;

(7) using nuclear transfer to create a first male animal that is autosomally isogenic, allelically isogenic and sexually non-isogenic to said haploid cell by fusing an isolated haploid cell or the nucleus therefrom selected from the expanded haploid cells of step (3) and the haploid cell or the nucleus therefrom from the haploid cell of step (7) with an enucleated metaphase II oocyte;

(8) using nuclear transfer to create a second animal that is autosomally isogenic, allelically isogenic and sexually isogenic to said haploid cell, by fusing an isolated cell in the G2 cell cycle phase (2n) or the nucleus therefrom selected from the expanded haploid cells of step (3) with an enucleated metaphase II oocyte;

wherein sexual reproduction between said first animal and said second animal produces offspring that are autosomally isogenic and allelically isogenic to said first and second animal.

Also included are methods of making an autosomally isogenic, allelically isogenic breeding pair of animals comprising:

(1) isolating a somatic cell from a preferred male animal;

(2) inducing meiosis to produce a haploid cell from said somatic cell;

(3) selecting a single haploid cell and determining whether it contains an X or Y chromosome;

(4) expanding said haploid cell;

(5) isolating a copy of said haploid cell or the nucleus therefrom;

(6) removing or the sex chromosome from said copy of said isolated haploid cell;

(7) inserting the alternative sex chromosome isolated from a non-isogenic animal or the original preferred animal or another haploid cell produced from said somatic cell and optionally expanding said haploid copy if an X chromosome is inserted;

(8) using nuclear transfer to create two animals that are autosomally isogenic, allelically isogenic and sexually non-isogenic, wherein

(a) the female animal is made by fusing two isolated haploid cells or the nuclei therefrom containing X chromosomes selected from the expanded haploid cells of step (4) or the expanded haploid cells of step (7) with an enucleated metaphase II oocyte in order to create one animal that has two X chromosomes OR by fusing one isolated haploid cell at the G2 cell cycle stage containing an X chromosome with an enucleated metaphase II oocyte in order to create one animal that has two X chromosomes; and

(b) the male animal is made by fusing one isolated haploid cell having an X chromosome with one isolated haploid cell having a Y chromosome with an enucleated metaphase II oocyte in order to create one animal that has an X and a Y chromosome;

wherein sexual reproduction between said first animal and said second animal produces offspring that are autosomally isogenic and allelically isogenic to said first and second animal.

The nuclear transfer units made by the methods of the present invention are also included. For instance, a female allelically isogenic diploid nuclear transfer unit may be made by a method comprising:

(1) isolating a somatic cell from a preferred female animal;

(2) inducing meiosis of said somatic cell by nuclear transfer of said somatic cell or the nucleus from said somatic cell (2n) into a metaphase II enucleated oocyte and activating said nuclear transfer unit to extrude a polar body (n), thereby resulting in a haploid activated nuclear transfer unit;

(3) allowing said haploid activated nuclear transfer unit to differentiate and expand to at least the two cell stage; and

(4) fusing either

(a) two haploid cells from step (3); or

(b) one haploid cell from step (3) at the G2 cell cycle stage;

with an enucleated metaphase II oocyte in order to create a female allelically isogenic diploid nuclear transfer unit. The method is performed such that the diploid nuclear transfer unit created at step (4) is activated such that there is no extrusion of a polar body. Activated diploid nuclear transfer units may further develop into an allelically isogenic cells, blastocysts, inner cell masses, ES cells, embryos, fetuses or animals.

Methods of making male autosomally isogenic, allelically isogenic diploid nuclear transfer units are also included, and such methods may be performed by:

(1) isolating a somatic cell from a preferred animal;

(2) inducing meiosis of said somatic cell by nuclear transfer of said somatic cell or the nucleus from said somatic cell (2n) into a metaphase II enucleated oocyte and activating said nuclear transfer unit to extrude a polar body (n), thereby resulting in a haploid activated nuclear transfer unit;

(3) allowing said haploid activated nuclear transfer unit to differentiate and expand to at least the two cell stage;

(4) replacing the sex chromosome in one cell from taken from said differentiated and expanded haploid cells using microcell-mediated chromosome transfer from the sex chromosome from a non-isogenic animal or from another haploid or somatic cell from said preferred animal if said preferred animal was a male;

(5) fusing two haploid cells:

(a) one from the expanded cells of step (3); and

(b) the cell made in step (4);

with an enucleated metaphase II oocyte in order to create a male autosomally isogenic, allelically isogenic diploid nuclear transfer unit. The allelically isogenic diploid nuclear transfer unit made by the methods of the invention are also encompassed.

Because the methods described herein enable one to pass the advantages of the cloning technology to the agricultural and other industries while at the same time enable the control over the dissemination of genetically engineered molecules to remain with the inventor or the assignee, the methods described herein are particularly useful business models. Accordingly, the invention also includes business methods for producing uniform, isogenic animals, comprising:

(1) producing autosomally isogenic and allelically isogenic male and female animals according to the methods described herein; and

(2) breeding said male and female animals to produce uniform, isogenic animals.

Female animals and/or said male animals may be genetically modified, bred or selected to provide an advantage in a desired market. For instance, in the agricultural market, female animals may be genetically modified, bred or selected to produce a high milk output, milk with specified lipid or protein profile, milk that contains a therapeutic protein, or milk with superior nutritional value. Alternatively, female and/or male animals may be genetically modified, bred or selected to produce meat, leather, wool or fiber having a desired characteristic.

Other target markets include laboratories, where there is a need for isogenic animals including rats, monkeys, rabbits, mice, guinea pigs to remove the statistical noise from experimentation and trials for the development of therapeutic drugs. A target market would also include a xenotransplantation facility, where animals such as cows, pigs and primates are developed to provide compatible organs for human transplantation. For instance, female animals and/or male animals may be genetically modified with a specific human HLA type profile, or modified such that native proteins that cause graft rejection are deleted, modified or replaced with proteins that do not cause graft rejection in humans.

One of the most effective business models is where the male animal has been genetically modified such that it only produces offspring of a single sex, i.e., such that it only produces female offspring. Such a model is useful where only female uniform, isogenic animals are sold commercially. Frozen semen from a male isogenic animal may also be isolated and sold to purchasers of female uniform, isogenic animals such that artificial insemination may be used to create further uniform, isogenic animals.

Male animals according to the invention may also be genetically modified such that they only produce male offspring, or such that they produce no offspring. This would be useful where only male uniform, isogenic animals are sold commercially. A single female isogenic animal could then sold or leased by purchasers of male uniform, isogenic animals such that purchasers may breed said female with a male in order to create further male uniform, isogenic animals.

The uniform, isogenic animals produced in the business methods described herein are also included in the invention, as is semen, and kits containing frozen semen for artificial insemination.

The skilled artisan will envision variations to the methods disclosed herein without departing from the scope of the invention.

### EXAMPLE 1

Isolation of Somatic Cells from Semen

The cloning of animals by nuclear transfer has many applications in such diverse fields as agriculture, medicine and the preservation of endangered species. One difficulty commonly faced, however, is an adequate source of somatic cells. In the case of agricultural species such as cattle, highly-valued studs are often lost with no known preservation of the genome for cloning. This invention describes a technique to isolate viable somatic cells from semen, urine, milk and other sources where the isolation of somatic cells is problematic.

While semen is often thought of as being largely a solution of spermatozoa that are haploid, somatic diploid cells may occasionally be shed as well. We centrifuged 0.75 ml of bovine semen at 700× g (45%-90% percoll gradient for 30 minutes), aspirated the supernatant, and resuspended the pellet of 500 ml in DMEM medium with 15 FCS. The resulting cell suspension was then plated in 35 mm² tissue culture plate. The culture dishes were aspirated, washed and refed 24 hours (after and every other day following). After five days of culture, fibroblastic cells were observed attached to the tissue culture dish. These somatic cells can then be propagated, cryopreserved, or used as somatic cell donors for the production of nuclear transfer embryos and calves. An alternative approach would be to use a Fluorescence Cell Sorter machine, which can separate sperm from somatic cells based upon DNA content.

To reduce the chance of spontaneous abortion, fetuses may be extracted at 40 days, and fetal fibroblasts isolated and frozen. From these fetal fibroblasts, the final animals can be cloned. Cells can be isolated in a similar manner from other fluids such as milk, blood or urine where such samples have been saved. In addition, such cells can be cultured from frozen tissue such as skin biopsy, skeletal muscle, or whole frozen animals.

The success of this method can be explained perhaps by analyzing the method of semen processing for the purpose of freezing and later use in artificial insemination. During extraction, an artificial vagina is used to collect the ejaculate and perhaps some of the cells that are around the penis along with free somatic cells originating in the accessory glands, ducts and testicle themselves will be mingled with the ejaculate. This technique will allow bulls to be “resurrected” in instances where the bulls are no longer alive but their frozen semen is available. The method is reproduced in detail below:

A. Establishment of Cell Lines from Cryopreserved Semen

NOTE: Please wear gloves for every step of the procedure to prevent cross contamination of samples

Percoll Separation of Sperm (Performed at Room Temperature)

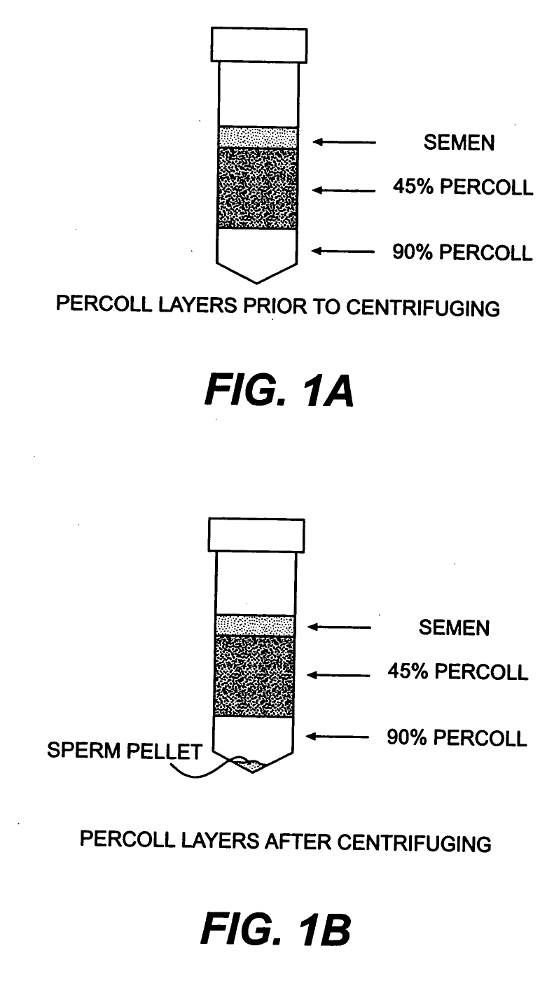

Step 1. In a sterile 15 ml conical centrifuge tube, layer 2 ml 90% Percoll then carefully layer 2 ml of 45% Percoll on top of the 2 ml of 90% Percoll layer as shown in the diagram below. It is best to use either a 1000 ul pipette or a 9 ml pastuer pippete. It is very critical to have a very defined interface between the two layers. This will be observed clearly because the 45% Percoll is pinkish in hue and the 90% Percoll is clear. A very defined interface will be observed if layered correctly (see FIG. 1A).

Step 2: Thaw semen in 35° C. water for 1 min. Record all information from semen straw, including bull name and registration numbers and collection date into your laboratory notebook. Step 3: Thoroughly dry the straw of semen with a KemWipe wet with ethanol and then snip end of semen straw with a clean scissor. Place the open end into a clean 15 ml conical tube. Then carefully snip off the plug end of the straw and deposit all semen into tube.

Step 4: With a 500 ul pipette, carefully layer all of the semen onto the top of the Percoll layers.

Step 5: Centrifuge at 700× g (2000 rpm using a 6.37 inch tip radius) for 30 minutes.

Step 6: After centrifugation, a sperm pellet will be observed at the bottom of the 90% Percoll layer as shown in diagram below (FIG. 1B).

Step 7: Aspirate off the Percoll gradients leaving the sperm pellet in the tip of the tube. This is usually about only 200 ul of pellet (this will vary depending on the number of semen straws thawed).

Step 8: With a clean pipette tip, move the pellet into either a 35 mm tissue culture treated plate or a 4 well Nunc plate with complete DMEM medium.

Step 9: Remove the medium the following day and add fresh medium to the plates.

Step 10: Carefully observe the plates for the presence of cells—this will depend on the semen, usually 7-14 days after the initial plating.

Step 11: Follow standard Cell Culture Techniques once a cell line is observed.

Stock Solutions

45% Percoll Solution

A. Ingredients

1. 1.5 ml 90% Percoll Stock Solution.

2. 1.5 ml Sperm TL with BSA.

B. Procedure

1. Use aseptic techniques.

2. Transfer ingredients to a sterile tube.

3. Invert to mix.

4. Do not attempt to filter.

Sperm TL Without BSA

A. Ingredients

1. 25 ml sperm TL stock.

2. Adjust pH to 7.4 with 1 M HCl.

3. Filter sterilize

4. Prepare daily.

Modified Sperm TL (10× Stock Used to Prepare 90% Percoll)

A. Ingredients

1. 3.09 ml 1M KCl.

2. 2.92 ml 0.1 M NaH₂PO₄

3. 4.675 gm NaCl

4. 2.380 gm Hepes

B. Procedure

1. Add prescribed amounts of KCl and NaH₂PO₄ solutions to ˜50 ml H₂O in volumetric flask.

2. Add NaCl and Hepes.

3. Adjust water to 100 ml.

4. Adjust pH to 7.3.

5. Filter sterilize and transfer to a glass bottle.

6. Store refrigerated indefinitely.

7. Readjust pH as needed.

1M CaCl₂—Used in Making 90% Percoll

A. Ingredients

1. 735 mg CaCl2*2H₂O.

2. Reagent grade water.

B. Preparation

1. Weigh CaCl₂.

2. Add 5 ml H₂O.

3. Filter sterilize or autoclave.

4. Store in glass bottle indefinitely.

0.1M MgCl₂—Used in Making 90% Percoll

A. Ingredients

1. 20.3 mg MgCl₂*6H₂O.

2. Reagent grade water.

B. Preparation

1. Weigh MgCl₂.

2. Add 10 ml water.

3. Filter sterilize or autoclave.

4. Store in glass bottle indefinitely.

90% Percoll Solution

A. Ingredients

1. 45.0 ml Percoll

2. 5.0 ml Modified Sperm T1 (10× stock)

3. 0.0985 ml 1M CaCl₂

4. 0.197 ml 0.1M MgCl₂

5. 0.184 ml Lactic Acid (60% syrup)

6. 104.5 mg NaHCO₃

B. Procedure

1. Combine ingredients while stirring.

2. Store refrigerated.

3. Do not attempt to filter.

1. SPERM TL STOCK

| | Final | | |
|---|---|---|---|
| Compound | mM | mg/100 ml | mg/500 ml |
| NaCl | 100 | 582 | 2910 |
| KCl | 3.1 | 23 | 115 |
| NaHCO₃ | 25 | 209 | 1045 |
| NaH₂PO₄H₂O | 0.29 | 4.1 | 20.5 |
| Hepes | 10 | 238 | 1190 |
| Na Lactate | 21.6 | 368 ul | 1840 ul |
| (60% syrup) | | | |
| Phenol Red | 1 ul/ml | 100 ul | 500 ul |
| CaCl₂2H₂O* | 2.10 | 29 | 145 |
| MgCl₂6H₂O* | 1.5 | 31 | 155 |

*Add last.

Check osmolarity (290-310 mOSM).

Filter into sterile bottle.

Store at 4° C.

Media components derived from: Parrish, J. J., J. L. Susko-Parrish and N. L. First. 1985. Theriogenology 24:537.

Chemical Components

| Sigma | | |
|---|---|---|
| Catalog | | |
| Number | Abbreviation | Name |
| C7902 | CaCl₂*2H₂O | Calcium Chloride-H₂O |
| H3375 | | Hepes |
| M2393 | MgCl₂—6H₂O | Magnesium Chloride-6H₂O |
| P1644 | | Percoll |
| P0290 | | Phenol Red |
| P5405 | KCl | Potassium Chloride |
| S5761 | NaHCO₃ | Sodium Bicarbonate |
| S5886 | NaCl | Sodium Chloride |
| L4263 | | Sodium Lactate (60% syrup) |
| S9638 | Na₂HPO₄*H₂O | Sodium Phosphate |

B. Nuclear Transfer Using Somatic Cells Isolated from Semen

Using the above techniques, we have found that when a single straw of semen is thawed and put in culture under conditions that will favor the growth of epithelial/fibroblast-like cells, colonies can be detected. Using this protocol, we were able to obtain somatic cells from a straw of bull semen, and use those somatic cells to generate embryos by nuclear transfer.

Three replicates of nuclear transfer were performed with three separate Londondale Sperm Cell Lines:

| Cultured | Cleaved | % Cleaved | Blastocysts | % Blastocysts |
|---|---|---|---|---|
| 51 | 26 | 51% | 9 | 18% |
| 191 | 73 | 38% | 37 | 19% |
| 49 | 28 | 57% | 10 | 20% |

6 embryos were transferred into three recipients, but no pregnancy was detected.

One replicate of nuclear transfer was performed with a Whiteleather Mark Sperm Cell Line.

| Cultured | Cleaved | % Cleaved | Blastocysts | % Blastocysts |
|---|---|---|---|---|
| 53 | 18 | 33% | 8 | 15% |

6 Embryos were transferred into 3 recipients—1 pregnancy was detected and is still ongoing (approx 67 days—sexed as male).

C. Characterization of Sperm Cell Lines

Karyotyping

Karyotypes were done on both sperm cell lines; images taken and saved. Results indicate that the cells are of bovine origin and have 60 chromosomes. Samples of NT embryos, cell line, semen and extracted DNA were sent to Celera AgGen for DNA analysis.

Staining of Semen Cell Line

Initial staining of cell lines was performed using alpha tubulin as a general (positive control) marker and Pan Cytokeratin as epithelium marker. Results indicated that there was no staining for the Pan Cytokeratin marker for both concentrations used. Alpha tubulin positive control worked (images not shown). This suggests that the cells are not of epithelial nor endothelial origin, and are probably fibroblasts.

What is claimed:

1. A method of altering the sex of a cloned animal, embryo, blastocyst, fetus or cell comprising:
(1) isolating a somatic or embryonic cell from an animal, embryo, blastocyst, fetus or other source of mammalian cells to be cloned;
(2) removing or programming for removal at least one sex chromosome from said somatic or embryonic cell;
(3) inserting at least one alternative sex chromosome from a non-isogenic animal; and
(4) using nuclear transfer to create an autosomally isogenic, sexually non-isogenic animal, embryo, blastocyst, fetus or cell.

2. The method of claim 1, wherein said at least one sex chromosome that is removed is a Y chromosome.

3. The method of claim 1, wherein said at least one sex chromosome that is removed is an X chromosome.

4. The method of claim 1, wherein said at least one alternative sex chromosome from a non-isogenic animal is from an allogeneic animal.

5. The method of claim 1, wherein said at least one alternative sex chromosome from a non-isogenic animal is from a xenogeneic species.

6. The method of claim 1 further comprising a step between steps (1) and (2) wherein said somatic or embryonic cell is propagated in culture prior to chromosome exchange.

7. The method of claim 6 further comprising a step whereby the propagated somatic or embryonic cells are genetically altered prior to chromosome exchange.

8. The method of claim 7, wherein said genetic alteration is a chromosomal insertion or deletion.

9. The method of claim 1, wherein said at least one alternative sex chromosome is genetically altered from its native state.

10. The method of claim 9, wherein said genetic alteration is a chromosomal insertion or deletion.

11. The method of claim 1, wherein said cloned animal is a mammal, fish, reptile or bird.

12. The method of claim 1 1, wherein said cloned animal is an ungulate selected from the group consisting of bovine, porcine, gaur, sheep and goat.

13. The method of claim 1, wherein said at least one alternative sex chromosome is inserted via microcell fusion or via injection.

14. The method of claim 5, wherein said cloned animal is a clone of an endangered species.

15. The method of claim 5, wherein said cloned animal is a clone of an extinct species.

16. A method of producing a sexual mate of an extinct animal, comprising:
(1) isolating a somatic or embryonic cell from said extinct animal;
(2) removing or programming for removal at least one sex chromosome from said somatic cell;
(3) inserting at least one alternative sex chromosome from a non-isogenic animal; and
(4) using nuclear transfer to create an autosomally isogenic, sexually non-isogenic animal.

17. The method of claim 16, wherein said somatic or embryonic cell is isolated from a sample of frozen cells.

18. The method of claim 17, wherein said frozen cells are frozen semen cells.

19. The method of claim 16, wherein said at least one alternative sex chromosome is from an allogeneic cell.

20. The method of claim 19, wherein said allogeneic cell is isolated from a sample of frozen cells.

21. The method of claim 16, wherein said at least one alternative sex chromosome is from a xenogeneic cell.

22. The method of claim 21, wherein said xenogeneic cell is from a species that is closely related to said extinct animal.

23. The method of claim 22, wherein said extinct animal is the buccardo mountain goat of Spain, and said closely related species is a domestic goat.

24. The method of claim 16, wherein said nuclear transfer generated sexual mate is carried by a surrogate female of a closely related species.

25. The method of claim 23, wherein said sexual mate of the buccardo is carried by a surrogate female domestic goat.

26. The method of clam 16, further comprising mating said sexual mate with a nuclear-transfer-generated clone of said extinct animal.

27. A method of sex selection, comprising:
(1) isolating a fertilized ovum, embryo or blastocyst;
(2) testing the sex of said ovum, embryo or blastocyst;
(3) removing or programming for removal the sex chromosome from one cell of said ovum, embryo or blastocyst if it is not of the desired sex;
(4) inserting the alternative sex chromosome isolated from an allogeneic animal;
(5) using nuclear transfer to create an autosomally isogenic, sexually non-isogenic embryo or blastocyst; and
(6) implanting said embryo or blastocyst into a surrogate female to isolate an animal having a desired sex.

28. The method of claim 27, wherein said allogeneic animal from which the alternative sex chromosome is isolated is the father of the ovum, embryo or blastocyst.

29. A method of eliminating chromosomal abnormalities from the clone of an animal, comprising;
(1) isolating a somatic or embryonic cell from an animal to be cloned;
(2) removing or programming for removal a damaged chromosome from said somatic or embryonic cell;
(3) inserting a non-damaged chromosome from a non-isogenic animal; and
(4) using nuclear transfer to create an animal, embryo, blastocyst, fetus or cell.

30. The method of claim 29, wherein said damaged chromosome has an inversion or translocation.

31. The method of claim 29, wherein said non-damaged chromosome is inserted via microcell fusion or via injection.

32. The method of claim 29, wherein said non-isogenic animal is allogeneic to said animal to be cloned.

33. The method of claim 11 wherein said cloned animal is a mammal, and said mammal is a mouse, hamster, guinea pig, primate or other laboratory animal.

34. The method of claim 11 wherein said cloned animal is a mammal, and said mammal is a cat, dog, horse or other companion animal.

35. A method of altering the sex of a cloned animal, embryo, blastocyst, fetus or cell comprising:
(1) isolating a nucleus from a somatic or embryonic cell of an animal to be cloned;
(2) removing or programming for removal at least one sex chromosome from said nucleus;
(3) inserting at least one alternative sex chromosome from a non-isogenic animal; and
(4) using nuclear transfer to create an autosomally isogenic, sexually non-isogenic animal, embryo, blastocyst, fetus or cell.

36. The method of claim 35, wherein said nucleus of a somatic or embryonic cell is preserved in a preservative prior to use in nuclear transfer.

37. The method of claim 36, wherein said preservative is alcohol.

38. The cloned animal, embryo, blastocyst, fetus or cell produced by the method of claim 1.

39. The sexual mate of an extinct animal produced by the method of claim 16.

40. The cloned animal of a desired sex produced by the method of claim 27.

41. A method of making an autosomally isogenic, allelically isogenic breeding pair of animals comprising:
(1) isolating a somatic cell from a preferred animal;
(2) inducing meiosis to produce a haploid cell from said somatic cell;
(3) making a diploid cell from said haploid cell which contains isogenic alleles;
(4) expanding said diploid cell;
(5) isolating a copy of said diploid cell or the nucleus therefrom;
(6) removing one sex chromosome from said copy of said isolated diploid cell;
(7) inserting the alternative sex chromosome from a non-isogenic animal;
(8) using nuclear transfer to create a first animal that is autosomally isogenic, allelically isogenic and sexually non-isogenic to said allelically isogenic diploid cell; and
(9) using nuclear transfer to create a second animal that is autosomally isogenic, allelically isogenic and sexually isogenic to said allelically isogenic diploid cell, wherein sexual reproduction between said first animal and said second animal produces offspring that are autosomally isogenic and allelically isogenic to said first and second animal.

42. A method of making an autosomally isogenic, allelically isogenic breeding pair of animals that only produces animals of a single sex, comprising steps (1)-(9) of claim 41 and further comprising a step or steps whereby a nucleic acid construct is introduced into at least one sex chromosome of the germ line of said male animal, wherein said nucleic acid construct encodes a transgene which is expressed post-meiotically in developing spermatids, and wherein expression of said transgene alters the fertility of sperm resulting from said developing spermatids, such that said male produces progeny of a single sex.

43. A method of producing autosomally isogenic and allelically isogenic offspring by sexual reproduction by mating the breeding pair of animals produced by the method of claim 41.

44. A method of producing autosomally isogenic, allelically isogenic and sexually isogenic offspring by sexual reproduction by mating the breeding pair of animals produced by the method of claim 42.

45. The breeding pair produced by the method of claim 41.

46. The breeding pair produced by the method of claim 42.

47. The offspring produced by the method of claim 43.

48. The offspring produced by the method of claim 44.

49. The method of claim 41, wherein said meiosis to produce a haploid cell is accomplished by nuclear transfer of said somatic cell or the nucleus from said somatic cell (2n) into a metaphase II enucleated oocyte, and activating said nuclear transfer unit to extrude a polar body (n), thereby resulting in a haploid activated nuclear transfer unit.

50. The method of claim 49, wherein said activation is accomplished by exposing said nuclear transfer unit to one or more treatments selected from the group consisting of hyaluronidase, ethanol, cytochalasin B, Ca²⁺ions, change in osmolarity, electrical pulse, bohemine, ionomycin and sperm factor.

51. The method of claim 49, wherein said activated haploid oocyte develops to at least the two cell stage.

52. The method of claim 51, wherein said allelically isogenic diploid cell is made by fusing two allelically isogenic haploid cells from said developing activated oocyte into an enucleated metaphase II oocyte.

53. A method of making an autosomally isogenic, allelically isogenic breeding pair of animals comprising:
(1) isolating a somatic cell from a preferred female animal;
(2) inducing meiosis to produce a haploid cell from said somatic cell;
(3) expanding said haploid cell;
(4) isolating a copy of said haploid cell or the nucleus therefrom;
(5) removing the X chromosome from said copy of said isolated haploid cell;
(6) inserting a Y chromosome isolated from a male animal;
(7) using nuclear transfer to create a first male animal that is autosomally isogenic, allelically isogenic and sexually non-isogenic to said haploid cell by fusing an isolated haploid cell or the nucleus therefrom selected from the expanded haploid cells of step (3) and the haploid cell or the nucleus therefrom from the haploid cell of step (7) with an enucleated metaphase II oocyte;
(8) using nuclear transfer to create a second animal that is autosomally isogenic, allelically isogenic and sexually isogenic to said haploid cell, by fusing two isolated haploid cells or the nuclei therefrom selected from the expanded haploid cells of step (3) with an enucleated metaphase II oocyte;
wherein sexual reproduction between said first animal and said second animal produces offspring that are autosomally isogenic and allelically isogenic to said first and second animal.

54. A method of making an autosomally isogenic, allelically isogenic breeding pair of animals comprising:
(1) isolating a somatic cell from a preferred male animal;
(2) inducing meiosis to produce a haploid cell from said somatic cell;
(3) selecting a single haploid cell and determining whether it contains an X or Y chromosome;
(4) expanding said haploid cell;
(5) isolating a copy of said haploid cell or the nucleus therefrom;
(6) removing the sex chromosome from said copy of said isolated haploid cell;
(7) inserting the alternative sex chromosome into said copy of said haploid cell wherein the alternative sex chromosome is isolated from either a non-isogenic animal or the original preferred animal or another haploid cell produced from said somatic cell and optionally expanding said haploid copy if an X chromosome is inserted;
(8) using nuclear transfer to create two animals that are autosomally isogenic, allelically isogenic and sexually non-isogenic by fusing isolated haploid cells or the nuclei therefrom from the expanded haploid cells of step (4) and/or the haploid cell or cells or the nuclei therefrom from the haploid cell or cells of step (7) with an enucleated metaphase II oocyte in order to create one animal that has two X chromosomes and one animal that has an X and a Y chromosome;
wherein sexual reproduction between said first animal and said second animal produces offspring that are autosomally isogenic and allelically isogenic to said first and second animal.

55. A method of making an autosomally isogenic, allelically isogenic breeding pair of animals comprising:
(1) isolating a somatic cell from a preferred female animal;
(2) inducing meiosis to produce a haploid cell from said somatic cell;
(3) expanding said haploid cell;
(4) isolating a copy of said haploid cell or the nucleus therefrom;
(5) removing the X chromosome from said copy of said isolated haploid cell;
(6) inserting a Y chromosome isolated from a male animal;
(7) using nuclear transfer to create a first male animal that is autosomally isogenic, allelically isogenic and sexually non-isogenic to said haploid cell by fusing an isolated haploid cell or the nucleus therefrom selected from the expanded haploid cells of step (3) and the haploid cell or the nucleus therefrom from the haploid cell of step (7) with an enucleated metaphase II oocyte;
(8) using nuclear transfer to create a second animal that is autosomally isogenic, allelically isogenic and sexually isogenic to said haploid cell, by fusing an isolated cell in the G2 cell cycle phase (2n) or the nucleus therefrom selected from the expanded haploid cells of step (3) with an enucleated metaphase II oocyte;
wherein sexual reproduction between said first animal and said second animal produces offspring that are autosomally isogenic and allelically isogenic to said first and second animal.

56. A method of making an autosomally isogenic, allelically isogenic breeding pair of animals comprising:
(1) isolating a somatic cell from a preferred male animal;
(2) inducing meiosis to produce a haploid cell from said somatic cell;
(3) selecting a single haploid cell and determining whether it contains an X or Y chromosome;
(4) expanding said haploid cell;
(5) isolating a copy of said haploid cell or the nucleus therefrom;
(6) removing or the sex chromosome from said copy of said isolated haploid cell;
(7) inserting the alternative sex chromosome isolated from a non-isogenic animal or the original preferred animal or another haploid cell produced from said somatic cell and optionally expanding said haploid copy if an X chromosome is inserted;
(8) using nuclear transfer to create two animals that are autosomally isogenic, allelically isogenic and sexually non-isogenic, wherein
(a) the female animal is made by fusing two isolated haploid cells or the nuclei therefrom containing X chromosomes selected from the expanded haploid cells of step (4) or the expanded haploid cells of step (7) with an enucleated metaphase II oocyte in order to create one animal that has two X chromosomes OR by fusing one isolated haploid cell at the G2 cell cycle stage containing an X chromosome with an enucleated metaphase II oocyte in order to create one animal that has two X chromosomes; and
(b) the male animal is made by fusing one isolated haploid cell having an X chromosome with one isolated haploid cell having a Y chromosome with an enucleated metaphase II oocyte in order to create one animal that has an X and a Y chromosome;
wherein sexual reproduction between said first animal and said second animal produces offspring that are autosomally isogenic and allelically isogenic to said first and second animal.

57. A method of making a female allelically isogenic diploid nuclear transfer unit comprising:
(1) isolating a somatic cell from a preferred female animal;
(2) inducing meiosis of said somatic cell by nuclear transfer of said somatic cell or the nucleus from said somatic cell (2n) into a metaphase II enucleated oocyte and activating said nuclear transfer unit to extrude a polar body (n), thereby resulting in a haploid activated nuclear transfer unit;
(3) allowing said haploid activated nuclear transfer unit to differentiate and expand to at least the two cell stage;
(4) fusing either
(a) two haploid cells from step (3); or
(b) one haploid cell from step (3) at the G2 cell cycle stage;
with an enucleated metaphase II oocyte in order to create a female allelically isogenic diploid nuclear transfer unit.

58. The method of claim 57, wherein said diploid nuclear transfer unit is activated such that there is no extrusion of a polar body.

59. The method of claim 58, wherein said activated diploid nuclear transfer unit further develops into an allelically isogenic cell, oocyte, blastocyst, inner cell mass, ES cell, embryo, fetus or animal.

60. The method of claim 58, wherein said activation is accomplished by exposing said nuclear transfer unit to one or more treatments selected from the group consisting of hyaluronidase, ethanol, cytochalasin B, Ca²⁺ ions, change in osmolarity, electrical pulse, bohemine, ionomycin and sperm factor.

61. A method of making a male autosomally isogenic, allelically isogenic diploid nuclear transfer unit comprising:
(1) isolating a somatic cell from a preferred animal;
(2) inducing meiosis of said somatic cell by nuclear transfer of said somatic cell or the nucleus from said somatic cell (2n) into a metaphase II enucleated oocyte and activating said nuclear transfer unit to extrude a polar body (n), thereby resulting in a haploid activated nuclear transfer unit;
(3) allowing said haploid activated nuclear transfer unit to differentiate and expand to at least the two cell stage;
(4) replacing the sex chromosome in one cell from taken from said differentiated and expanded haploid cells using microcell-mediated chromosome transfer from the sex chromosome from a non-isogenic animal or from another haploid or somatic cell from said preferred animal if said preferred animal was a male;
(5) fusing two haploid cells:
(a) one from the expanded cells of step (3); and
(b) the cell made in step (4);
with an enucleated metaphase II oocyte in order to create a male autosomally isogenic, allelically isogenic diploid nuclear transfer unit.

62. The method of claim 61, wherein said diploid nuclear transfer unit is activated such that there is no extrusion of a polar body.

63. The method of claim 60, wherein said activated diploid nuclear transfer unit further develops into an allelically isogenic cell, oocyte, blastocyst, inner cell mass, ES cell, embryo, fetus or animal.

64. The method of claim 63, wherein said activation is accomplished by exposing said nuclear transfer unit to one or more treatments selected from the group consisting of hyaluronidase, ethanol, cytochalasin B, Ca²+ions, change in osmolarity, electrical pulse, bohemine, ionomycin and sperm factor.

65. The allelically isogenic diploid nuclear transfer unit made by the method of claim 57.

66. The allelically isogenic diploid nuclear transfer unit made by the method of claim 61.

67. The allelically isogenic cell, oocyte, blastocyst, inner cell mass, ES cell, embryo, fetus or animal made by the method of claim 59.

68. The allelically isogenic cell, oocyte, blastocyst, inner cell mass, ES cell, embryo, fetus or animal made by the method of claim 63.

69. The breeding pair produced by the method of claim 53.

70. The breeding pair produced by the method of claim 54.

71. The breeding pair produced by the method of claim 55.

72. The breeding pair produced by the method of claim 56.

73. The offspring produced by mating the breeding pair of claim 53.

74. The offspring produced by mating the breeding pair of claim 54.

75. The offspring produced by mating the breeding pair of claim 55.

76. The offspring produced by mating the breeding pair of claim 56.

77. A business method for producing uniform, isogenic animals, comprising:
(1) producing autosomally isogenic and allelically isogenic male and female animals using the method of claim 1; and
(2) breeding said male and female animals to produce uniform, isogenic animals.

78. The business method of claim 77, wherein said female animal and/or said male animal is genetically modified, bred or selected to provide an advantage in a desired market.

79. The business method of claim 78, wherein said desired market is agriculture, and said isogenic animals include cows, pigs, sheep, goats, birds and fish.

80. The business method of claim 79, wherein said female animal is genetically modified, bred or selected to produce a high milk output, milk with specified lipid or protein profile, milk that contains a therapeutic protein, or milk with superior nutritional value.

81. The business method of claim 79, wherein said female animal and/or male animal is genetically modified, bred or selected to produce meat, leather, wool or fiber having a desired characteristic.

82. The business method of claim 78, wherein said desired market comprises laboratories, and said isogenic animals include rats, monkeys, rabbits, mice and guinea pigs.

83. The business method of claim 78, wherein said desired market includes xenotransplantation facilities, and said animals include cows, pigs and primates.

84. The business method of claim 83, wherein said female animal and/or said male animal is genetically modified with a specific human HLA type profile, or modified such that native proteins that cause graft rejection are deleted, modified or replaced with proteins that do not cause graft rejection in humans.

85. The business method of claim 77, wherein the male animal is genetically modified such that it only produces offspring of a single sex.

86. The business method of claim 85, wherein the male animal is genetically modified such that it only produces female offspring.

87. The business method of claim 86, wherein only female uniform, isogenic animals are sold commercially.

88. The business method of claim 87, wherein frozen semen from a male isogenic animal is isolated and sold to purchasers of female uniform, isogenic animals such that artificial insemination may be used to create further uniform, isogenic animals.

89. The business method of claim 88, wherein the frozen semen that is sold to said purchasers is from a male uniform, isogenic animal that has been genetically modified such that it produces only female offspring when used for artificial insemination.

90. The business method of claim 85, wherein the male animal is genetically modified such that it only produces male offspring.

91. The business method of claim 90, wherein only male uniform, isogenic animals are sold commercially.

92. The business method of claim 91, wherein a female isogenic animal is sold to purchasers of male uniform, isogenic animals such that purchasers may breed said female with a male in order to create further uniform, isogenic animals.

93. The business method of claim 90, wherein said male offspring are genetically modified, bred or selected to provide an advantage in the beef or pig market.

94. The uniform, isogenic animals produced in the business method of claim 77.

95. The frozen semen isolated in the business method of claim 88.

96. The male animal produced in the business method of claim 86.

97. The male animal produced in the business method of claim 90.

98. The method of claim 41, wherein said alternative sex chromosome is inserted via microcell-mediated chromosomal transfer.

99. The method of claim 98, wherein said alternative sex chromosome is genetically modified with a selectable marker.

100. The method of claim 99, wherein said selectable marker is aminoglycosde phosphotransferase.

101. The method of claim 41, wherein the sex chromosome to be removed is genetically modified in said somatic cell to facilitate removal.

102. The method of claim 101, wherein said genetic modification facilitating removal is a marker gene that can be negatively selected.

103. The method of claim 101, wherein said genetic modification facilitating removal is an insertion of a gene or DNA sequence that results in loss of the chromosome upon mitotic division.

104. The method of claim 103, wherein said inserted DNA sequence is a centromere sequence.

105. The method of claim 53, wherein said alternative sex chromosome is inserted via microcell-mediated chromosomal transfer or injection.

106. The method of claim 105, wherein said alternative sex chromosome is genetically modified with a selectable marker.

107. The method of claim 106, wherein said selectable marker is aminoglycosde phosphotransferase.

108. The method of claim 53, wherein the X chromosome to be removed is genetically modified in said somatic cell to facilitate removal.

109. The method of claim 108, wherein said genetic modification facilitating removal is a marker gene that can be negatively selected.

110. The method of claim 108, wherein said genetic modification facilitating removal is an insertion of a gene or DNA sequence that results in loss of the chromosome upon mitotic division.

111. The method of claim 110, wherein said inserted DNA sequence is a centromere sequence.
